(19) United States
(12) Patent Application Publication
(10) Pub. No.: US 2010/0069317 A1
(43) Pub. Date: Mar. 18, 2010

(54) CRYSTALLINE NEMORUBICIN HYDROCHLORIDE

(75) Inventors: Romualdo FORINO, Milan (IT); Natale Alvaro BARBUGIAN, Milan (IT); Attilio TOMASI, Milan (IT); Giuseppina VISENTIN, Rho (IT); Marco CATTANEO, Milan (IT); Massimo ZAMPIERI, Cesano Maderno (MI) (IT)

Correspondence Address: SCULLY SCOTT MURPHY & PRESSER, PC, 400 GARDEN CITY PLAZA, SUITE 300, GARDEN CITY, NY, 11530, US

(73) Assignee: NERVIANO MEDICAL SCIENCES S.R.L., Nerviano (MI) (IT)

(21) Appl. No.: 12/373,391

(22) PCT Filed: Jun. 29, 2007
(86) PCT No.: PCT/EP2007/056592

§ 371 Date: Jan. 12, 2009

(30) Foreign Application Priority Data
Jul. 12, 2006 (EP) 06117013.0

(51) Int. Cl. A61K 31/704; C07H 15/24; A61P 35/00

(52) U.S. Cl. 514/34; 536/6.4

(57) ABSTRACT

This invention relates to a novel crystalline polymorphic form of nemorubicin hydrochloride dihydrate, useful for the preparation of pharmaceutical composition for the treatment of tumors. A process for preparing this novel polymorphic form, named form A, is within the scope of the present invention.

### SUMMARY OF THE INVENTION

This invention relates to a novel crystalline polymorphic form of nemorubicin hydrochloride, an antitumor drug. A process for preparing this novel polymorphic form is within the scope of the present invention.

### BACKGROUND OF THE INVENTION

Nemorubicin hydrochloride, chemical names (8S-cis,2″S)-7,8,9,10-tetrahydro-6,8,11-trihydroxy-8-(hydroxyacetyl)-1-methoxy-10-{[2,3,6-trideoxy-3-(2-methoxy-4-morpholinyl)-α-L-lyxo-hexopyranosyl]oxy}-5,12-naphthacenedione hydrochloride and 3′ des amino-3′ [2(S)methoxy-4-morpholinyl] doxorubicin-hydrochloride (below referred to as nemorubicin hydrochloride only) of formula

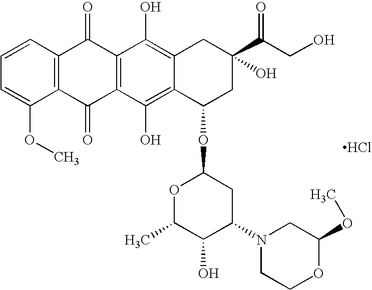

is a doxorubicin derivative obtained with the substitution of the —NH₂ at position 3′ in the sugar moiety with a methoxymorpholino group. The compound was synthesized in the course of a research program aimed at identifying new anthracyclines with at least partially novel modes of action, and possessing broad spectrum of activity, including activity on multidrug resistant (mdr) tumors. U.S. Pat. No. 4,672,057 discloses and claims nemorubicin, preparation process, pharmaceutical compositions and medical uses thereof. Vasey et al., Cancer Research, Vol. 55, No. 10, 1995, pages 2090-2096, describes phase I clinical and pharmacokinetic studies with nemorubicin administered by intravenous (i.v.) bolus injection in patients with refractory solid tumors including patients with liver metastases from colorectal cancer.

Nemorubicin is active in vitro and in vivo on experimental tumors resistant to anthracyclines, vinca alkaloids and taxaned (mdr phenotype). In addition, no cross-resistance was observed on tumor cells resistant to alkylating agents (such as melphalan and cisplatin), or on cells resistant to topoisomerase II inhibitors (such as etoposide). Nemorubicin is active after intraperitoneal, i.v. or oral administration, with good antitumor activity on murine leukemias, and on solid murine and human tumor models.

The compound differs from most anthracyclines in being highly potent when administered in vivo, the optimal i.v. dose being at least 80 fold less than that of doxombicin. This result, and the observation that the cytotoxic activity of nemorubicin is increased in vitro in the presence of mouse, rat and human liver microsomes, suggests that nemorubicin may be transformed into highly cytotoxic metabolite(s).

The high lipophilicity of the molecule, which confers to the compound the ability to reach high intracellular concentrations and is most likely one of the reasons of its efficacy on resistant models, makes it effective also after oral administration. The oral treatment with nemorubicin is associated, in all the animal models examined, with an antitumor activity comparable to that observed after i.v. administration at doses 1.3-2 fold higher than the effective i.v. doses. In particular, in liver metastases from M5076 murine fibrosarcoma, the best result (doubling of survival time) was achieved with the oral administration. This might be a reflection of a different behavior of the drug, due to first pass effect to the liver. In addition, the liver is a common site of metastasis in many human cancers. Nemorubicin represents a therapeutic option in the treatment of a liver cancer. According to WO 00/15203, nemorubicin can be administered via the hepatic artery, for example, as an infusion of from about 15 minutes to about 30 minutes every 4 weeks or preferably, as a 5-10 minute bolus every 4-8 weeks, to adult patients with either a hepatic metastatic cancer, for example, patients with colorectal cancer who have progressed after receiving i.v. chemotheraphy or intrahepatic 5-fluorouracil or 5-fluorodeoxyuridine (FUDR) chemotheraphy, or patients with primary liver carcinoma such as, for example, hepatocellular carcinoma or cholangiocarcinorna involving the liver. According to WO 00/15203, nemorubicin can be administered to a patient in a dosage ranging from, e.g., about 100 mcg/m² to about 1000 mcg/m², preferably from about 100 mcg/m²to about 800 mcg/m², for example, in a dosage of about 200 mcg/m². WO 04/75904 describes and claims the use of nemorubicin for the preparation of a medicament for the treatment of a human liver tumor, which comprises intrahepatic administration of nemorubicin via the hepatic artery in a dosage ranging from, e.g., about 100 mcg/m² to about 800 mcg/m², preferably from about 200 mcg/m² to about 600 mcg/m², for example in a dosage of about 200, 400 or 600 mcg/m² every 6 weeks. Two administration schedules have been evaluated in Phase I setting: in one trial nemorubicin was administered by IBA as a 30-minute infusion every 4 weeks (q4w) in saline; in another trial, nemorubicin was administered by IBA with iodinated oil as a 5 to 10 minute infusion every 6-8 weeks (q6-8w).

As described in WO 04/082579 and WO 00/066093, nemorubicin is indicated as a component of therapy in combination with radiotherapy, alkylating agent, an antimetabolite, a topoisomerase I/II inhibitor or a platinum derivative.

Suarato et al., ACS Symposium Series (1995), 574 (Anthracycline Antibiotics), pages 142-55 and U.S. Pat. No. 5,304,687 disclose key intermediates and processes for an improved synthesis of nemorubicin hydrochloride.

Polymorphism is the property of some molecules to adopt more than one crystalline form in the solid state. A single molecule may give rise to a variety of solids having distinct physical properties that can be measured in a laboratory like its thermal behavior, e.g. melting point and differential scanning calorimetry (“DSC”) thermogram, dissolution rate, X-ray diffraction pattern, infrared absorption spectrum. The differences in the physical properties of polymorphs result from the orientation and intermolecular interactions of adjacent molecules in the bulk solid. Accordingly, polymorphs are distinct solids sharing the same molecular formula which may yet have distinct advantageous and/or disadvantageous physical properties compared to other forms in the polymorph family.

### SUMMARY OF THE INVENTION

It is an object of the invention to provide a novel polymorphic crystalline form of nemorubicin hydrochloride and a methods of its manufacture. A further object is the use of the polymorph for the preparation of formulations and related compositions thereof intended for the i.v., intrahepatic or oral administration.

These and other objects are provided by one or more of the embodiments described below. One embodiment of the invention provides a polymorphic crystalline form of nemorubicin hydrochloride dihydrate characterized by having an X-ray powder diffraction pattern comprising reflection peaks at the following 2θ angle values of about 6.4, 9.3, 105, 11.4, 11.9, 12.2, 12,7, 12.9, 13.1, 15.1, 15.4, 16.3, 17.3, 19.1, 19.4, 20.2, 20.9, 21,2, 21.5, 22.5, 22.9, 23.6, 24.1, 24,3, 25.5, 26.0, 27.4, 28.5 and 28.8.

The polymorph can provide an X-ray powder diffraction pattern substantially in accordance with that shown in FIG. 1. The crystalline form was also characterized by its DSC, as shown in the thermogram of FIG. 2, with endothermic peak related to melting with decomposition in the range 170-200° C. Another embodiment of the invention provides a process for preparing the polymorph. The method comprises standing for a period of time a solution of nemorubicin hydrochloride as amorphous, in an alcohol. Another aspect relates to samples crystalline nemorubicin hydrochloride dihydrate having a % purity >85%, preferably >96%.

A fourth embodiment of the invention provides the use of polymorph nemorubicin hydrochloride dihydrate crystalline form in the preparation of formulations intended for i.v., intrahepatic or oral administration, as well as such resultant formulations.

### BRIEF DESCRIPTION OF THE DRAWING

The invention is also illustrated by reference to the accompanying drawings described below.

FIG. 1 shows powder X-ray diffractogram of crystalline polymorph of nemorubicin hydrochloride dihydrate, prepared as described in example 1.

FIG. 2 shows DSC thermogram of crystalline polymorph of nemorubicin hydrochloride dihydrate, prepared as described in example 1. Heat flow values are along the Y-axis, temperatures along the X-axis.

### DETAILED DESCRIPTION OF THE INVENTION

It has now been found that nemorubicin hydrochloride may exist in crystalline polymorphic form containing two molecules of water. This novel crystalline form is fully characterized herein below and is referred to, for convenience, as “Form A”. Owing to its crystalline properties, the new Form A of nemorubicin hydrochloride dihydrate according to the invention has surprising advantages with regard to the amorphous in terms of stability and process ability. As a matter of fact, an amorphous substance is more hygroscopic and much less chemically stable than a crystalline one. Crystalline substances are easier to be handled than amorphous substances with particular regard when amorphous form is highly hygroscopic such as nemorubicin amorphous is. In particular, new Form A of nemorubicin hydrochloride dihydrate allows also its formulation into pharmaceutical forms intended for the oral route. As a matter of fact, high hygroscopicity and amorphous state are features not compatible with the design and realization of plain formulations intended for the oral route of administration such as capsules and tablets, due to the difficulties in handling the active drug substance in terms of homogeneity within formulation blends, changes in its physico-chemical properties along the formulation stages due to the severe absorption of water, difficulties in maintaining the chemical and physical stability of the formulations themselves with ageing due to the natural chemical instability of hygroscopic amorphous substances.

Even more a crystallization step is a very good way to enhance chemical purity without the use of costly techniques such as chromatography.

No prior art of which applicants are aware describes Form A as now provided herein.

To the best of applicants' knowledge, Form A of the invention is previously unknown and not suggested by the art.

It is therefore an object of the present invention a new crystalline form of nemorubicin hydrochloride dihydrate, which is here below referred as Form A.

The absolute intensity (CPS—counts per second) and relative intensity (%) of the characteristics reflection peaks of Form A at the 2θ angle values are reported in the following TABLE I.

TABLE I

| Position | Intensity | Relative Intensity |
|---|---|---|
| 2θ (Deg.) ± 0.2 | (CPS) | (%) |
| 6.4 | 1267.72 | 100.00 |
| 9.3 | 77.85 | 6.14 |
| 10.5 | 69.21 | 5.46 |
| 11.4 | 426.84 | 33.67 |
| 11.9 | 176.88 | 13.95 |
| 12.2 | 286.58 | 22.61 |
| 12.7 | 548.43 | 43.26 |
| 12.9 | 216.07 | 17.04 |
| 13.1 | 275.82 | 21.76 |
| 15.1 | 45.78 | 3.61 |
| 15.4 | 94.69 | 7.47 |
| 16.3 | 120.75 | 9.52 |
| 17.3 | 378.10 | 29.82 |
| 19.1 | 84.58 | 6.67 |
| 19.4 | 177.86 | 14.03 |
| 20.2 | 33.78 | 2.66 |
| 20.9 | 89.84 | 7.09 |
| 21.2 | 106.04 | 8.36 |
| 21.5 | 52.69 | 4.16 |
| 22.5 | 294.56 | 23.24 |
| 22.9 | 36.84 | 2.91 |
| 23.6 | 46.81 | 3.69 |
| 24.1 | 158.38 | 12.49 |
| 24.3 | 100.99 | 7.97 |
| 25.5 | 104.82 | 8.27 |
| 26.0 | 202.26 | 15.95 |
| 27.4 | 55.16 | 4.35 |
| 28.5 | 64.92 | 5.12 |
| 28.8 | 27.94 | 2.20 |

Form A was characterized with a principal reflection peak (100% of relative intensity) at 6.4 deg (2θ).

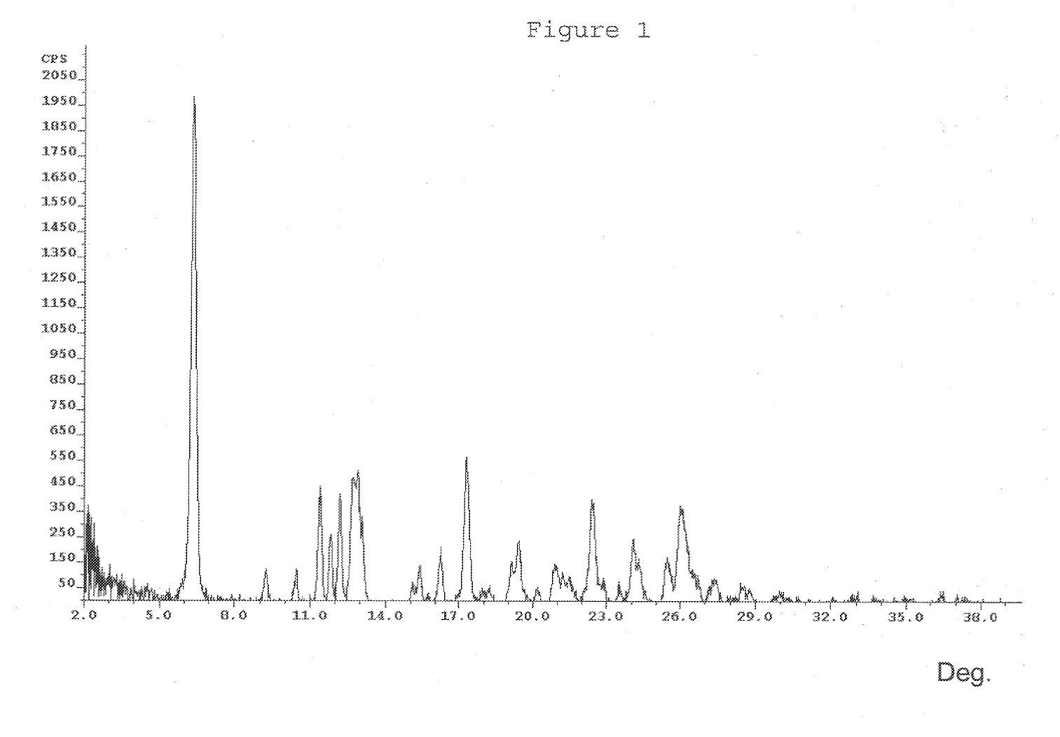

In particular, the Form A polymorph is characterized by an X-ray powder diffraction spectrum substantially in accordance with that shown in FIG. 1.

Modification in relative intensity may occur according to particular properties of the particles (e.g. size, aggregation) without indicating a modification of the crystal form. Moreover, instrument variation and other factors may affect the 2θ values; therefore, the peak assignments may vary by plus or minus 0.2°.

Differential Scanning Calorimetry

The DSC thermogram of the Form A showed an initial broad endotherm related to desolvation (up to 140° C.) followed by endothermic peak related to melting with decomposition in the range 170-200° C.

This behaviour is clearly distinguishable from the one of the amorphous.

The Form A and amorphous of nemorubicin hydrochloride may be readily distinguished by X-ray powder diffraction and DSC.

What differentiates form A towards the amorphous form of nemorubicin is its behaviour when exposed to humidity. The crystalline form A of nemorubicin hydrochloride dihydrate in fact is non hygroscopic. This specific beneficial property allows a more convenient manufacturing of the final drug, in particular oral formulations that are not subject to instability of the active drug substance both along the manufacturing process and subsequently when formulations are subject to stability studies. Owing to its crystalline properties, Form A of nemorubicin hydrochloride dihydrate according to the invention possesses greater stability than the previously known amorphous form, which makes the Form A more suitable for preparing the final drug in any formulation, including the oral ones.

As a matter of fact, the hygroscopicity of the Form A is much lower than that one of the amorphous, as shown in the following table II:

TABLE II

| | |
|---|---|
| Relative Humidity | Change In Mass (%) |

| (%) | Form A | Amorphous |
|---|---|---|
| 0.0 | 0.00 | 0.00 |
| 3.1 | 1.92 | 0.30 |
| 6.2 | 3.53 | 0.83 |
| 9.3 | 3.82 | 1.35 |
| 12.4 | 4.02 | 1.93 |
| 15.5 | 4.17 | 2.45 |
| 18.6 | 4.29 | 2.92 |
| 21.7 | 4.39 | 3.37 |
| 24.8 | 4.49 | 3.79 |
| 27.9 | 4.57 | 4.19 |
| 31.0 | 4.65 | 4.62 |
| 34.1 | 4.71 | 5.08 |
| 37.2 | 4.77 | 5.52 |
| 40.3 | 4.82 | 5.96 |
| 43.5 | 4.86 | 6.43 |
| 46.6 | 4.91 | 6.90 |
| 49.7 | 4.95 | 7.37 |
| 52.8 | 5.01 | 7.88 |
| 55.9 | 5.07 | 8.36 |
| 59.0 | 5.13 | 8.92 |
| 62.1 | 5.19 | 9.46 |
| 65.2 | 5.25 | 10.10 |
| 68.3 | 5.31 | 10.85 |
| 71.4 | 5.38 | 11.69 |
| 74.5 | 5.46 | 12.67 |
| 77.6 | 5.52 | 14.01 |
| 80.7 | 5.58 | 15.76 |
| 83.8 | 5.66 | 18.09 |
| 86.9 | 5.75 | 21.51 |
| 90.0 | 5.85 | 26.66 |

The above-tabulated data provide strongly evidence of the different hygroscopicity between the known amorphous and Form A of the present invention, stable in its dihydrate form.

The crystalline form A of the present invention, when subject to a desorption cycle, loses the two moles of water. The same amount of water is promptly re-adsorbed when the RH of the atmosphere reaches 30%. The change in mass of about 5% at about 55% RH indicates that the form A is a dihydrate even though a slight water excess is absorbed at higher RH.

Therefore, the different hygroscopicity profile of the crystalline material allows for a much higher stability of the moisture content upon handling and storage. As a consequence, considering working conditions, the possibility to work with a stable material allows the transformation of the active drug substance in the final dosage form without technical difficulties. Even more, the handling of a crystalline material versus an amorphous one allows easier blending, tabletting, capsule filling processes.

The invention also provides a process for preparing the above Form A according to the invention.

Specifically, crystalline nemorubicin hydrochloride dihydrate can be produced by dissolving amorphous nemorubicin hydrochloride in an alcoholic solution, optionally partially removing the solvent from the solution at a temperature of up to about 25° C., optionally under vacuum, and crystallizing nemorubicin hydrochloride at a temperature of from 0° to 30° C., preferably at room temperature. Preferably the solution of nemorubicin hydrochloride is kept under inert atmosphere, more preferably under nitrogen. Suitable alcohols include methanol, ethyl alcohol and mixture thereof. The amount of the alcohol dissolving nemorubicin hydrochloride is, for example, 1 to 50 parts by weight per part of nemorubicin hydrochloride. Preferably, the amount of alcohol may be 1 to 20 parts by weight, more preferably I to 10 parts by weight per part of nemorubicin hydrochloride.

If it is necessary to partially remove the solvent, the temperature of the solution of nemorubicin hydrochloride may be, for example, up to 30° C., more preferably of from 20° to 30° C. The solution from which nemorubicin hydrochloride is crystallized is held at a temperature of 0° to 30° C. during the crystallization, preferably at room temperature. The period of time for crystallizing the nemorubicin hydrochloride is not limited, but preferably it is in the range of 15 to 30 days. More preferably, the process comprises standing from 20 to 22 days the solution of nemorubicin hydrochloride (as amorphous) in methanol.

Seed crystals of crystalline nemorubicin hydrochloride dihydrate may be added into the solution to accelerate crystallization,

The thus obtained crystals may be recovered by common procedures, for example by filtration under reduced pressure or by centrifugal filtration, followed by drying the crystals. The drying treatment can be carried out in a conventional manner, for example by subjecting the crystals to a reduced pressure at a temperature of from 0° to 30° C., preferably from 15 to 25° C., more preferably at room temperature. The pressure in drying may be, for example, less than 200 mmHg, preferably 1 to 50 mmHg. The drying treatment can be monitored by measuring the solvent amount in the crystals. Usually, the drying will be completed in 1 to 48 hours. The dried product is then placed in presence of room humidity, RH about 40-60%, preferably RH 45-50%, for a period of time from 5′ to 1 hour, preferably from 15 to 30 minutes, even more preferably for about 20′, so as to obtain the crystalline nemorubicin hydrochloride dihydrate of the present invention

Crystalline nemorubicin hydrochloride dihydrate may be also prepared by subjecting amorphous nemorubicin hydrochloride to a procedure analogous to that described above.

Analytical Methods

X-Ray Powder Diffraction

X-ray powder diffraction analyses were performed using a Thermo/ARL XTRA apparatus based on Bragg-Brentano geometry with a Cu Kα generator working at 45 KV/40 mA (1.8 kW power) and a Peltier-cooled solid-state detector. The spectral range was from 2 to 40 2θ, explored with a single continuous scan acquisition at a rate of 1.2 degree/min (steps of 0.020° and acquisition time of 1 second/step. The sample was loaded on a low background silicon plate by flattening the powder on its surface by gently pressing with a flat spatula. The obtained patterns were reported in intensity (CPS—counts per second) vs. 2 0 (two-theta) angle (Deg) charts.

### Hygroscopicity

Hygroscopicity tests were performed by means of a DVS 1000 apparatus (SMS) allowing dynamic water vapour sorption analysis. Multiple sorption/desorption cycles between 0% and 90% RH were performed at 25° C.

The equipment is a “controlled atmosphere microbalance” where the weighed sample is exposed to controlled variations of the relative humidity (RH) at a constant temperature.

### Differential Scanning Calorimetry

Differential Scanning Calorimetry analysis was carried out with a Perkin-Elmer DSC-7 apparatus. Aluminum DSC pans (volume of 50 μL with holes) were loaded with 2÷4 mg of sample. An aluminum disc was placed over the powder in order to obtain a thin layer and improve thermal exchange.

The sample was analyzed at least in duplicate under nitrogen flow at a heating rate of 10° C/min over the range 30-250° C.

Indium, Tin and Lead (LOC certified reference materials) were used to assess the calibration of the apparatus with regard to the temperature scale and the enthalpy response. The starting materials for preparing Form A, can be obtained by a variety of procedures well known to those of ordinary skill in the art. For example, nemorubicin hydrochloride as amorphous can be prepared by the general procedure taught by the above-cited US patents.

The following examples illustrate but do not limit the scope of the invention.

### Example 1

Preparation of Nemorubicin Hydrochloride Dihydrate (Form A)

1.0 g of nemorubicin hydrochloride amorphous, prepared as described in U.S. Pat. No. 5,304,687, was dissolved in 10 ml of methanol at room temperature.

The mixture was left for 20 days at room temperature and then filtered. The product was dried in vacuo at 20-25° C. for 18 hours and then placed in a chamber in presence of humidity (RH 40-50%) for 20 minutes.

0.6 g of Form A of nemorubicin hydrochloride dihydrate were obtained.

### Example 2

Nemorubicin hydrochloride dihydrate formulation in capsule can be prepared with common fillers and excipients. Compositions for the 1 mg and 2.5 mg unit dosage strengths are here below presented.

### Example 2a

1 mg strength

| | A | B |
|---|---|---|
| Nemorubicin HCl 2H₂O | 0.67% | 0.74% |
| Mannitol | 99.33% | |
| Pregelatinized Starch | | 99.26% |
| Final filling weight | 150 mg | 135 mg |

Hard gelatin capsule size: 4

### Example 2b

2.5 mg strength

| | A | B |
|---|---|---|
| Nemorubicin HCl 2H₂O | 1.00% | 1.14% |
| Mannitol | 99.00% | |
| Pregelatinized Starch | | 98.86% |
| Final filling weight | 250 mg | 220 mg |

Hard gelatin capsule size: 2

Experimental batches were prepared using mortar and pestle with batch size of about 25-50 g. Required amount of active ingredient and an amount of filler equivalent in volume were passed through 400-500 μm net and gently blended into the mortar. Then aliquot of filler equivalent in volume to the mortar content was added and pestle mixing continued. The previous step was continued until the addition of filler was completed. The final blend obtained was distributed into hard gelatin capsules.

### Example 3

To increase dose flexibility, formulations useful for automatic capsules filling process are prepared starting from prototypes described in the above example. Lubricant is added to avoid sticking to pistons of dosing tubes. Application of volumetric dosing tubes allows use of single formulation to prepare capsules with different strengths. Two formulations are shown covering strength ranges between 1-4 mg and 2.5-10 mg respectively.

### Example 3a

| | 1-4 mg strengths | | | |
|---|---|---|---|---|
| | A | B | C | D |
| Nemorubicin HCl 2H₂O | 1.00% | 1.00% | 1.00% | 1.00% |
| Mannitol | 98.50% | | | 98.25% |
| Pregelatinized Starch | | 98.00% | 98.25% | |
| Stearic acid | 0.50% | | | |
| Glyceril behenate | | 1.00% | | |
| Glyceril palmitostearate | | | | 0.75% |
| Sodium stearyl fumarate | | | 0.75% | |

The capsule size and filling weight corresponding to different strengths are listed in the following table

| Strength | 1.0 mg | 2.0 mg | 2.5 mg | 3.0 mg | 4.0 mg |
|---|---|---|---|---|---|
| Filling weight | 100 mg | 200 mg | 250 mg | 300 mg | 400 mg |
| Hard gelatin capsule size | 4 | 3 | 2 | 1 | 0 |

### Example 3b

| | 2.5-10 mg strengths | | | |
|---|---|---|---|---|
| | A | B | C | D |
| Nemorubicin HCl 2H₂O | 2.50% | 2.50% | 2.50% | 2.50% |
| Mannitol | 97.00% | | | 96.75% |
| Pregelatinized Starch | | 96.50% | 96.75% | |
| Stearic acid | 0.50% | | | |
| Glyceril behenate | | 1.00% | | |
| Glyceril palmitostearate | | | | 0.75% |
| Sodium stearyl fumarate | | | 0.75% | |

The capsule size and filling weight corresponding to different strengths are listed in the following table

| Strength | 2.5 mg | 5.0 mg | 7.5 mg | 10.0 mg |
|---|---|---|---|---|
| Filling weight | 100 mg | 200 mg | 300 mg | 400 mg |
| Hard gelatin capsule size | 4 | 3 | 1 | 0 |

### Example 4

Nemorubicin Hydrochloride Dihydrate Formulation in Tablets

Starting from compositions proposed in the above examples, formulation for Nemorubicin hydrochloride dihydrate tablets can be defined. In this case lactose, mannitol and pregelatinized starch grade is suitable for direct compression process, microcrystalline cellulose is added to improve compressibility and the amount of lubricant is slightly increased to reduce sticking risk to punches and help ejection from dies.

### Example 4a

| | 1-4 mg strengths | | | |
|---|---|---|---|---|
| | A | B | C | D |
| Nemorubicin HCl 2H₂O | 1.00% | 1.00% | 1.00% | 1.00% |
| Pregelatinized starch | | 75.50% | | |
| Mannitol | 80.00% | | 87.50% | 72.50% |
| Microcrystalline cellulose | 17.50% | 22.00% | 10.00% | 25.00% |
| Glyceryl behenate | 1.50% | | | 1.50% |
| Glyceryl palmitostearate | | 1.50% | 1.50% | |

| Strength | 1.0 mg | 2.0 mg | 3.0 mg | 4.0 mg | 2.0 mg |
|---|---|---|---|---|---|
| Tablet weight | 100.0 mg | 200 mg | 300 mg | 400 mg | 200 mg |

### Example 4b

| | 2.5-10 mg strengths | | | |
|---|---|---|---|---|
| | A | B | C | D |
| Nemorubicin HCl 2H₂O | 2.50% | 2.50% | 2.50% | 2.50% |
| Pregelatinized starch | | 74.50% | | |
| Mannitol | 79.00% | | 86.50% | 71.50% |
| Microcrystalline cellulose | 17.00% | 21.50% | 9.50% | 24.50% |
| Glyceryl behenate | 1.50% | | | 1.50% |
| Glyceryl palmitostearate | | 1.50% | 1.50% | |

| Strength | 2.5 mg | 5.0 mg | 7.5 mg | 10.0 mg | 5.0 mg |
|---|---|---|---|---|---|
| Tablet weight | 100 mg | 200 mg | 350 mg | 400 | 200 mg |

1. A crystalline nemorubicin hydrochloride.

2. A crystalline nemorubicin hydrochloride containing two molecules of water.

3. A crystalline nemorubicin hydrochloride dihydrate according to claim 2 having an X-ray powder diffraction pattern comprising reflection peaks at the following 2θ angle values of about 6.4, 9.3, 10.5, 11.4, 11.9, 12.2. 12.7, 12.9, 13.1, 15.1, 15.4, 16.3, 17.3, 19.1, 19.4, 20.2, 20.9, 21.2, 21.5, 22.5, 22.9, 23.6, 24.1, 24.3, 25.5, 26.0, 27.4, 28.5 and 28.8.

4. A crystalline nemorubicin hydrochloride dihydrate according to claim 3 having the distinctive peaks in the powder X-ray diffraction shown in the following table 1:
TABLE I

| Position | Intensity | Relative Intensity |
|---|---|---|
| 2θ (Deg.) ± 0.2 | (CPS) | (%) |
| 6.4 | 1267.72 | 100.00 |
| 9.3 | 77.85 | 6.14 |
| 10.5 | 69.21 | 5.46 |
| 11.4 | 426.84 | 33.67 |
| 11.9 | 176.88 | 13.95 |
| 12.2 | 286.58 | 22.61 |
| 12.7 | 548.43 | 43.26 |
| 12.9 | 216.07 | 17.04 |
| 13.1 | 275.82 | 21.76 |
| 15.1 | 45.78 | 3.61 |
| 15.4 | 94.69 | 7.47 |
| 16.3 | 120.75 | 9.52 |
| 17.3 | 378.10 | 29.82 |
| 19.1 | 84.58 | 6.67 |
| 19.4 | 177.86 | 14.03 |
| 20.2 | 33.78 | 2.66 |
| 20.9 | 89.84 | 7.09 |
| 21.2 | 106.04 | 8.36 |
| 21.5 | 52.69 | 4.16 |
| 22.5 | 294.56 | 23.24 |
| 22.9 | 36.84 | 2.91 |
| 23.6 | 46.81 | 3.69 |
| 24.1 | 158.38 | 12.49 |
| 24.3 | 100.99 | 7.97 |
| 25.5 | 104.82 | 8.27 |
| 26.0 | 202.26 | 15.95 |
| 27.4 | 55.16 | 4.35 |
| 28.5 | 64.92 | 5.12 |
| 28.8 | 27.94 | 2.20 |

5. A crystalline nemorubicin hydrochloride dihydrate according to claim 2 having the powder X-ray diffraction spectrum of FIG. 1.

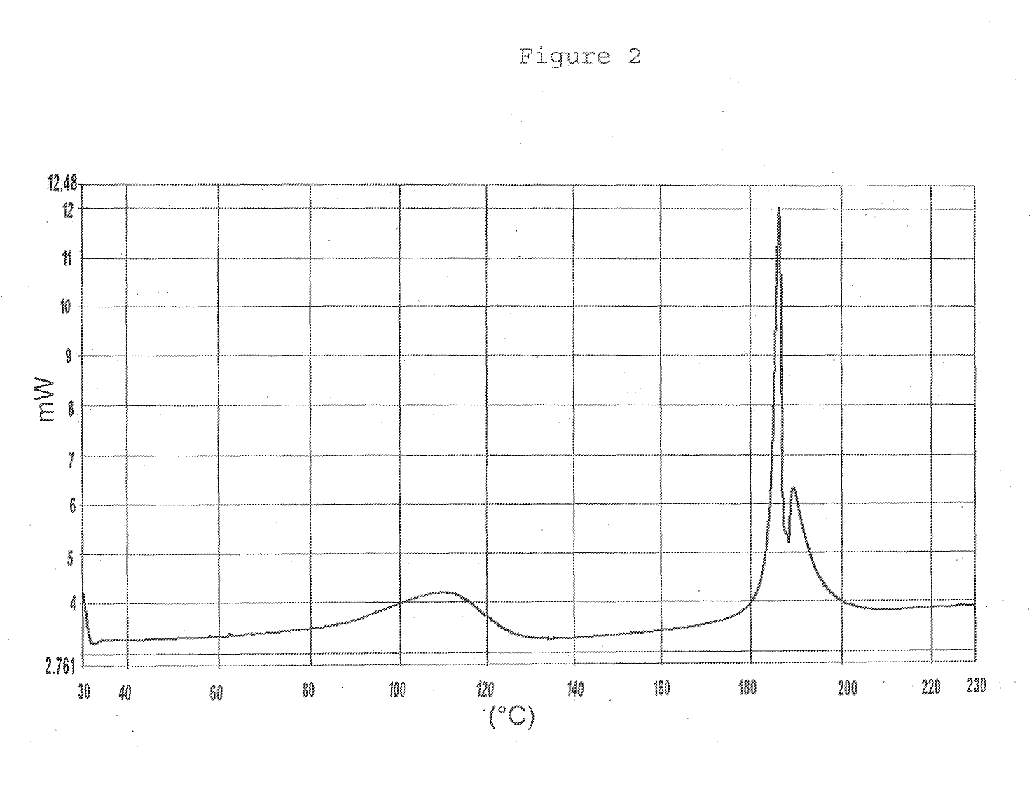

6. A crystalline nemorubicin hydrochloride dihydrate according to claim 2 having the DSC thermogram shown in FIG. 2.

7. A crystalline nemorubicin hydrochloride dihydrate according to claim 2 having a % purity >85% or >96%.

8. A process for preparing a crystalline nemorubicin hydrochloride according to claim 1 or 2 comprising crystallizing nemorubicin hydrochloride.

9. A process according to claim 8 comprising:
dissolving amorphous nemorubicin hydrochloride in an alcohol and crystallizing nemorubicin hydrochloride at a temperature of from 0° to 30° C. and
recovering and drying the resultant crystals.

10. A process according to claim 9 in which the alcohol is methanol, ethyl alcohol or a mixture thereof.

11. A process according to claim 8 characterized in that it comprises standing from 20 to 22 days a solution of amorphous nemorubicin hydrochloride in methanol.

12. A process for preparing a crystalline nemorubicin hydrochloride dehydrate according to claim 2 comprising crystallizing amorphous nemorubicin hydrochloride as described in claims 9 to 11, and placing the dried product in the presence of room humidity.

13. A crystalline nemorubicin hydrochloride according to claim 1 or 2 for use in the treatment of the human or animal body by therapy.

14. (canceled)

15. A pharmaceutical composition comprising a crystalline nemorubicin hydrochloride according to claim 1 or 2 and a pharmaceutically acceptable diluent or carrier.

16. A pharmaceutical composition according to claim 15 suitable for oral administration.

17. A method of treating cancer comprising administrating to a person in need thereof, a pharmaceutical composition according to claim 15.
